(19) United States
(12) Patent Application Publication
(10) Pub. No.: US 2024/0317667 A1
(43) Pub. Date: Sep. 26, 2024

(54) Method and Plant for the Production of Vinyl Acetate

(71) Applicant: LINDE GMBH, Pullach (DE)

(72) Inventors: Ricardo Bermejo De Val, München (DE); Martin Schubert, München (DE); Andreas Meiswinkel, Rimsting (DE); Ernst Haidegger, Riemerling (DE)

(21) Appl. No.: 18/577,190

(22) PCT Filed: Jul. 6, 2022
(86) PCT No.: PCT/EP2022/068799

§ 371 Date: Jan. 5, 2024

(30) Foreign Application Priority Data
Jul. 6, 2021 (EP) 21020352.7

(51) Int. Cl. C07C 67/05; C07C 5/48; C07C 51/215

(52) U.S. Cl. CPC C07C 67/05; C07C 5/48; C07C 51/215

(57) ABSTRACT

The present invention proposes coupling an oxidative dehydrogenation with a vinyl acetate synthesis, wherein the vinyl acetate synthesis is fed with ethylene and acetic acid from the oxidative dehydrogenation. A common carbon dioxide removal is provided.

### CROSS-REFERENCE TO RELATED APPLICATIONS

This application is the national phase of, and claims priority to, International Application No. PCT/EP2022/068799 filed Jul. 6, 2022, which claims priority to European Application No. EP 21020352.7, filed Jul. 6, 2021.

### FIELD OF THE INVENTION

The invention relates to a process and to a system for producing vinyl acetate.

### BACKGROUND

Oxidative dehydrogenation (ODH) of paraffins having two to four carbon atoms is generally known. During the ODH, said paraffins are converted with oxygen, inter alia, to give the respective olefins and water. The invention relates in particular to the oxidative dehydrogenation of ethane to ethylene, hereinafter also referred to as ODHE.

ODH(E) may be advantageous over more established methods for producing olefins, such as steam cracking or catalytic dehydrogenation. For instance, due to the exothermic nature of the reactions involved and the practically irreversible formation of water, there is no thermodynamic equilibrium limitation. The ODH(E) can be carried out at comparatively low reaction temperatures. In principle, no regeneration of the catalysts used is required, since the presence of oxygen enables or causes regeneration in situ. Finally, in contrast to steam cracking, lower amounts of valueless by-products, such as coke, are formed.

For further details regarding ODH(E), reference may be made to the relevant literature, for example: Ivars, F. und López Nieto, J. M., Light Alkanes oxidation: Targets Reached and Current Challenges, in: Duprez, D. und Cavani, F., Handbook of Advanced Methods and Processes in oxidation Catalysis: From Laboratory to Industry, London 2014: Imperial College Press, Pages 767-834, or Gärtner, C. A. et al., Oxidative Dehydrogenation of Ethane: Common Principles and Mechanistic Aspects, ChemCatChem, Vol. 5, no. 11, 2013, pages 3196 to 3217, and X. Li, E. Iglesia, Kinetics and Mechanism of Ethane Oxidation to Acetic Acid on Catalysts Based on Mo—V—Nb Oxides, J. Phys. Chem. C, 2008, 112, 15001-15008.

In particular, MoVNb-based catalyst systems have shown promise for ODH(E), as mentioned, for example, in F. Cavani et al, “Oxidative dehydrogenation of ethane and propane: How far from commercial implementation?”, Catal. Today, 2007, 127, 113-131. Catalyst systems additionally containing Te can also be used. Where reference is made herein to a “MoVNb-based catalyst system” or a “MoVTeNb-based catalyst system”, this shall be understood to mean a catalyst system which has the elements mentioned as a mixed oxide, also expressed as MoVNbOₓ or MoVTeNbOₓ, respectively. Where Te is given in parentheses, this indicates its optional presence. The invention is used in particular with such catalyst systems.

During the ODH, particularly when MoVNb(Te)Oₓ-based catalysts are used under industrially relevant reaction conditions, significant amounts of the respective carboxylic acids of the paraffins used, in particular acetic acid in the case of ODHE, are formed as by-products. For economical system operation, a co-production of olefins and the carboxylic acids is generally unavoidable when using the type of catalyst described; a preferred formation of olefins is accordingly usually desirable in technical applications.

Depending on the reaction conditions and catalysts used, in ODH(E), in addition to the by-products already mentioned (wherein acetic acid can also represent a further product of value, for example), certain amounts of carbon monoxide and carbon dioxide are also formed as undesired by-products. Current improvement and optimization approaches are partially aimed at minimizing the proportion of these carbon oxides. In this case, the proportion of carbon oxides can be pushed back to less than 5%.

Recently, commercial concepts have become known based on the earlier, rather theoretical approaches and/or work on a laboratory scale, which are in particular based on the mentioned mixed oxide catalysts. For example, reference can be made here to EP 3 519 377 B1 (WO 2018/115416 A1), WO 2018/115418 A1, WO 2018/082945 A1, U.S. Pat. No. 10,730,810 B2 (WO 2018/115494 A1), EP 3 700 663 B1 (WO 2019/081682 A1), WO 2019/243480 A1, U.S. Pat. No. 10,730,811 B2 (WO 2018/115414 A1), WO 2020/187572 A1 of the applicant, and WO 2018/153831A1, WO 2018/019761 A1 WO 2018/019760 A1, WO 2017/198762 A1, WO 2017/144584 A1, WO 2020/074750 A1, U.S. Pat. No. 9,963,412 B2, U.S. Pat. No. 10,017,432 B2 of other applicants. What these concepts have in common is that air or oxygen is always used as an oxidizing agent, and only small amounts of carbon oxides are contained in the product stream.

With regard to subsequent process steps, ODHE usually aims for a high purity of the ethylene product (e.g., so-called “polymer grade”, in particular more than 99.9%). Therefore, in particular, suitable purification steps and removal of trace components downstream of the actual ODHE, in particular residual contents of oxygen from ODHE, and carbon monoxide and acetylene compounds formed, must be considered.

For the removal of acetylene from a process gas from ethylene plants (steam crackers), for example, isothermal crude gas hydrogenations (after drying, before a known C2/C3 separation, i.e., deethanization), isothermal C2 hydrogenations (after C2/C3 separation, before C1/C2 separation, i.e., demethanization) or adiabatic tail-end hydrogenations (usually after C1/C2 cleavage and before or after C2 splitters) are used. For details, reference is made to technical literature, such as the article “Ethylene” in Ullmann's Encyclopedia of Industrial Chemistry, online edition, 2009, DOI 10.2002/14356007.a10_045.pub3.

In the established steam cracking technology, the hydrogenations mentioned are all present in the absence of molecular oxygen and at carbon monoxide contents of significantly less than 1% (typically less than 2,500 ppm, in particular less than 1,500 ppm). While in particular Ni-based catalysts were originally used for this purpose, noble metal catalysts (in particular Pd-based) are used nowadays for such concepts, which can additionally be doped with further metals, such as Ag, Ce, inter alia. Likewise, in particular in the scientific literature, the use of Rh, Ir, Ru, Pt and Au-based systems is reported.

The presence (comparatively) of high carbon monoxide contents, as in the case of ODH(E), therefore usually requires other approaches. A combined removal of carbon monoxide and oxygen directly at the outlet of an ODHE reactor was thus described for example in US 2010/0256432 A1 and WO 2017/144584 A1.

EP 3 708 557 A1 and EP 3 708 558 A1 describe a process and suitable catalysts which enable at least partial removal of acetylene and/or oxygen from the product gas of an ODHE. In particular, copper oxide or ruthenium-containing catalysts are advantageously used. This acetylene and/or oxygen removal can be followed by carbon dioxide removal. In a corresponding process, the process gas, or a gaseous mixture which is formed using at least a portion of the process gas, can be partly or completely subjected to, in the order indicated here, a condensate separation, a compression, an at least partial removal of the oxygen and of one or more of the acetylenes, and one or more stages of a carbon dioxide removal, the at least partial removal of the oxygen and of the acetylene or acetylenes taking place simultaneously and by catalytic reaction using a catalyst containing copper oxide or ruthenium, or in the form of a hydrogenation.

According to WO 2018/153831 A1, oxygen, carbon monoxide, and optionally acetylene are removed from the product stream of an ODHE. In this case, it is generally disclosed to use an oxidation catalyst which, as components, can in particular have the metals Ni, Cu, Zn, Pd, Ag, Pt, Au, Fe, Mn, Ce, Sn, Ru and Cr. In this case, Cu and/or Pt-based systems, but in particular Cu-based systems, are preferably used. In the previously cited WO 2017/144584A1, the same applicant describes a concept for oxygen removal as a downstream bed in an ODH main reactor, i.e., before the process condensate separation, this downstream bed being heated/cooled with a separate coolant stream. The catalyst of this downstream bed can, on the one hand, have the same composition as the ODH catalyst or can be modified by other elements such as Sb, but preferably Cu.

In addition to the requirement for the high product purity of the ethylene product, downstream cryogenic system parts also require a quantitative separation of carbon dioxide. Owing to its strong interaction with suitable solvents or scrubbing liquids, carbon dioxide-particularly in concentrations as arise in the ODHE—can likewise be removed comparatively easily from the product mixture, wherein it is possible to use known methods for removing carbon dioxide, in particular corresponding scrubbing (for example amine scrubbing). The laden scrubbing liquid is then regenerated in a separate column, and very pure carbon dioxide is released by desorption.

Should subsequent steps require the absence of, or only a very low residual concentration of, carbon dioxide (for example due to catalytic inhibition or poisoning), the residual carbon dioxide content after amine scrubbing can be further reduced by an optional caustic scrubbing as fine cleaning, as required.

With certain exceptions, the corresponding scrubbing liquids can also react with oxygen, as a result of which disadvantageous aging or damage to the scrubbing agents, which require a continuous purge and makeup stream or lead to an undesired shortening of the service life of these scrubbing liquids, can occur over time. Therefore, from this point of view, the removal of oxygen upstream of a corresponding scrubbing is advantageous.

According to the prior art, water is typically removed by means of a regenerative molecular sieve-based dryer and, in addition to achieving the product specification, said water removal is likewise mandatory with regard to subsequent cryogenic process steps in order to avoid distortions due to deposition of ice.

Typically, in the context of a suitable procedure, methane contained in the product stream must also be removed in an ODHE process (e.g., in particular from the ethane feed stream of the ODHE). A C1/C2 separation (“demethanizer” or demethanization) is usually used for this purpose, which requires corresponding cryogenic conditions. Such a demethanizer simultaneously removes carbon monoxide from the product stream. A C1 minus fraction is thus formed, which contains methane and/or carbon monoxide as essential components. In general, a “C1 minus fraction” is to be understood here as a fraction that contains predominantly or exclusively methane and components that are volatile at lower temperature than methane, such as, in particular, carbon monoxide and oxygen. Accordingly, a “C2 plus fraction” is a fraction that predominantly or exclusively contains components with higher boiling points than methane. The term “predominantly or exclusively” or “rich” is used to denote a proportion of at least 75%, 80%, 85%, 90%, 95% or 99% based on volume, mass or moles. Corresponding numerical values also apply if a “predominant portion” is mentioned, for example in relation to any product or following streams. A “predominant portion” refers in particular to more than 50%, more than 66%, more than 75% or more than 90% (relative to a partial volume of a total volume or a subset of a total quantity and the like). If a fraction is referred to by the name of the main component contained therein (“ethane fraction”, “ethylene fraction” and the like), it does not necessarily have to consist exclusively of the same, but can also predominantly comprise this component or be rich in this component in the sense explained.

Traces of oxygen still contained in the inlet flow of the demethanizer also arrive in this C1 minus fraction. It is therefore usually the goal to limit the inlet content of oxygen in the inlet stream of the demethanizer, and thus to avoid the possible formation of an explosive atmosphere at the top of a column used here (cf. also WO 2018/082945 A1). EP 3 456 703 A1 describes, for example, a demethanizer in the decomposition part of an ODHE system, which is (also) combined with pressure swing adsorption in the top stream for this purpose.

Ultimately, unreacted ethane has to be separated from ethylene, which takes place by means of a C2 splitter, which is likewise operated under cryogenic conditions. This must thus be constructed and operated such that ethane in the ethylene is virtually quantitatively removed, and at the same time an ethane stream, which is recirculated to the ODHE, contains as little ethylene as possible or no ethylene.

A main application for vinyl acetate (VAM) is the polymerization to polyvinyl acetate (PVAc) or to other copolymers. Vinyl acetate thus represents an important intermediate for the chemical industry.

The production process customary nowadays starts from ethylene and acetic acid and is described, for example, in the article “Vinyl Esters” in Ullmann's Encyclopedia of Industrial Chemistry, online edition, 2019, DOI 10.1002/14356007.a27_419.pub2. These are reacted in an exothermic reaction with the admixture of oxygen in a gas phase process. Fixed bed reactors with Pd catalysts are usually used. In particular, the oxidative conversion of ethylene to carbon dioxide is a relevant side reaction. Ethylene and acetic acid are preferably used in a ratio of 2 to 3 to 1, and oxygen is added in a ratio of up to 0.5 equivalents with respect to acetic acid. Typical reaction conditions are 120° C. to 180° C. at a pressure of 5 bar to 12 bar. Typically, an inert gas, such as carbon dioxide (for example 10 to 30%), nitrogen or argon is added in order to better control the heat of reaction and ignition regions. In principle, it is also possible to synthesize vinyl acetate without adding carbon dioxide, or with a significantly lower proportion of carbon dioxide than mentioned above.

Impurities in the feed stream of the vinyl acetate synthesis, in particular acetylene and carbon monoxide, have a negative effect on vinyl acetate synthesis. The feed for the vinyl acetate synthesis is therefore ideally free of these compounds or contains them only in extremely low concentrations. A suitable ethylene feed for the vinyl acetate synthesis can therefore be provided according to the prior art in particular as highly purified ethylene (e.g. “polymer grade”) from corresponding processes, for example steam cracking and a downstream purification and decomposition part.

The actual synthesis of vinyl acetate is then followed downstream by processing, which in particular comprises a phase separation (separation of gas phase and liquid crude product) and advantageously also a scrubbing, which, in particular, serves to increase the efficiency of the yield of vinyl acetate by means of extraction with acetic acid—but which is also the basis of a recovery of ethylene and optionally carbon dioxide as starting materials for the synthesis of the vinyl acetate. In most cases, a separate absorptive removal of carbon dioxide is then carried out in this stream or a substreams in order to be able to adjust the carbon dioxide content. In the aforementioned article in Ullmann's Encyclopedia of Industrial Chemistry, in particular a regenerative potassium carbonate solution is listed for this purpose. For the subsequent purification of the vinyl acetate raw product, various distillative solutions are used, which are explained in detail in the article mentioned. The treatment (phase separation, scrubbing, absorptive removal of carbon dioxide and distillation) is usually carried out at a lower pressure than the synthesis of the vinyl acetate.

In principle, approaches for an integration of the ODHE and vinyl acetate synthesis into one process are known. These appear fundamentally advantageous since ODHE usually delivers acetic acid as a co-product, and vinyl acetate synthesis in turn starts from ethylene and acetic acid.

### SUMMARY

An object of the invention is to demonstrate improved and more effective possibilities for the integration of the ODHE and vinyl acetate synthesis into one process.

According to one embodiment of the invention, a method for producing vinyl acetate, comprising: in one or more first process steps: subjecting ethane and oxygen are subjected to an oxidative catalytic dehydrogenation to obtain a first product mixture containing ethane, ethylene, water, acetic acid and carbon dioxide; and in one or more second process steps: subjecting ethylene and acetic acid are subjected to a catalytic vinyl acetate synthesis to obtain a gaseous second product mixture containing ethylene, vinyl acetate, and carbon dioxide. At least a portion of the ethylene and at least a portion of the acetic acid which are subjected to the catalytic reaction in the one or more second process steps are formed by at least a portion of the ethylene and at least a portion of the acetic acid from the first product mixture. The method further includes forming a first subsequent mixture containing ethane, ethylene and carbon dioxide using at least a portion of the first product mixture. The formation of the first subsequent mixture comprises an at least partial removal of oxygen, carbon monoxide and/or acetylene, and further comprises subjecting at least a portion of the first product mixture and of the first subsequent mixture to a condensing deposition of water and acetic acid to obtain a condensate fraction containing water and acetic acid. At least a portion of the acetic acid which is subjected to the catalytic vinyl acetate synthesis in the one or more second process steps is formed by at least a portion of the acetic acid from the condensate fraction. The method continues by forming a second subsequent mixture containing ethylene and carbon dioxide using at least a portion of the second product mixture. The formation of the second subsequent mixture comprises subjecting at least a portion of the second product mixture to a vinyl acetate separation to obtain a precursor fraction containing vinyl acetate and the second subsequent mixture. At least most of the first subsequent mixture and the second subsequent mixture are combined to form a third subsequent mixture containing ethane, ethylene and carbon dioxide. At least a portion of the third subsequent mixture is subjected to one or more third process steps. The one or more third process steps comprise a carbon dioxide removal. A fourth subsequent mixture containing ethane and ethylene, and a carbon dioxide fraction are formed in the one or more third process steps. At least a portion of the fourth subsequent mixture is subjected to a separation in which an ethane fraction and an ethylene fraction are formed. Finally, at least a part of the fourth subsequent fraction and/or at least a part of the ethylene fractions is subjected to a demethanization. At least a portion of the ethylene which is subjected to the catalytic vinyl acetate synthesis in the one or more second process steps is formed by at least a portion of the ethylene from the ethylene fraction, and/or at least a portion of the ethane which is subjected to the oxidative catalytic dehydrogenation in the one or more first process steps is formed by at least a portion of the ethane from the ethane fraction. At least a part of the fourth subsequent fraction and/or at least a part of the ethylene fraction is subjected to a demethanization.

A system for implementing the method is further described herein.

### BRIEF DESCRIPTION OF THE DRAWINGS

FIGS. 1 to 4 illustrate processes and systems on the basis of which features according to the invention are explained, in the form of schematic process diagrams, wherein

FIG. 2 refers to a variant not forming part of the invention.

### WRITTEN DESCRIPTION

In the following, known processes which comprise an integration of the vinyl acetate synthesis and ODHE in one process will be explained in order to distinguish them with respect to advantages and features of the invention.

Where pressure values are indicated below using the “bar” unit, they refer to absolute pressures, unless mentioned otherwise. Pressure differences are expressed in bar.

WO 2018/114747 A1 discloses, inter alia, a removal of carbon dioxide and a C2 splitter downstream of the vinyl acetate synthesis and carbon dioxide removal, with recycling of ethylene into the vinyl acetate synthesis and ethane into the ODHE. However, at least one substream from the ODHE—which also contains carbon dioxide due to the process—is always incorporated into the vinyl acetate synthesis. A bypass of the vinyl acetate synthesis is the only thing which allows direct transport of a substream from the ODHE into the carbon dioxide removal. Such a design means that only a limited possibility of varying the carbon dioxide content is possible. In particular, if there are changes in the reaction behavior and product distribution behavior of the ODHE, undesired changes can occur. In particular, therefore, the complete separation of the carbon dioxide in the inlet stream of the vinyl acetate synthesis is not possible, and/or enrichment to only very low residual contents of carbon dioxide is possible. In addition, no information is included regarding the advantageous design of the corresponding pressure levels of the individual reaction and separation steps, and a corresponding, necessary compression of the process streams, and in particular return streams. In particular, this document suggests a decreasing pressure gradient, with carbon dioxide removal taking place at lower pressure than the vinyl acetate synthesis.

WO 2018/114752 A1 also discloses an integration of the ODHE and vinyl acetate synthesis. The focus there is the use of a vapor permeation for separating water from the outlet stream of the ODHE. Embodiments of the further process control are otherwise strongly based on the aforementioned document, and also do not disclose any further solutions in the sense of the present invention. None of the two aforementioned documents is based on the presence of methane in the process streams (in particular resulting from methane in the feed of the ODHE), and no approaches for methane separation are thus disclosed.

In particular, these documents also do not relate to the influence of trace components, in particular carbon monoxide and acetylene, on the vinyl acetate synthesis. A solution for removing these trace components is not proposed.

WO 1998/005620 A1, which describes a sequential interconnection of the ODHE and vinyl acetate synthesis, is cited in the aforementioned documents. However, this document also focuses in particular on the separation and optionally recirculation of acetic acid and/or vinyl acetate or correspondingly enriched substreams. This document does not contain specific statements regarding a carbon dioxide removal. The outlet stream of the ODHE is usually fed to the vinyl acetate synthesis without a carbon dioxide removal. There is therefore no removal, depletion, or adaptation of the carbon dioxide content in the inlet stream of the vinyl acetate synthesis. This document also does not mention the presence of methane in the process streams, and thus also does not contain any approach for methane separation in an integrated process.

According to a variant disclosed in WO 1998/005620 A1, carbon monoxide can be oxidized in the ODHE to form carbon dioxide. Necessary for this purpose, however, is a catalyst which can form an ODHE product stream with very low proportions of carbon monoxide. As described above, typical ODHE catalysts, in particular MoVNb-based catalyst systems, do not (yet) fulfill this requirement. Thus, this approach does not disclose a solution for sufficient carbon monoxide removal in the sense of an integration of the ODHE and vinyl acetate synthesis. According to a further variant, a Pd-containing catalyst is used in the vinyl acetate synthesis, which also converts carbon monoxide in a suitable reaction. However, as described in the document, this design is limited to Pd-based catalysts. Even if the catalysts currently used for the vinyl acetate synthesis are typically Pd-based, the additional carbon dioxide removal and coupling of the reaction steps in the vinyl acetate synthesis results in a significant limitation in the process parameters—for example, where there are changes in the composition of the inlet stream-since the behaviors of the carbon monoxide removal and of the vinyl acetate synthesis cannot be altered and adjusted independently of one another.

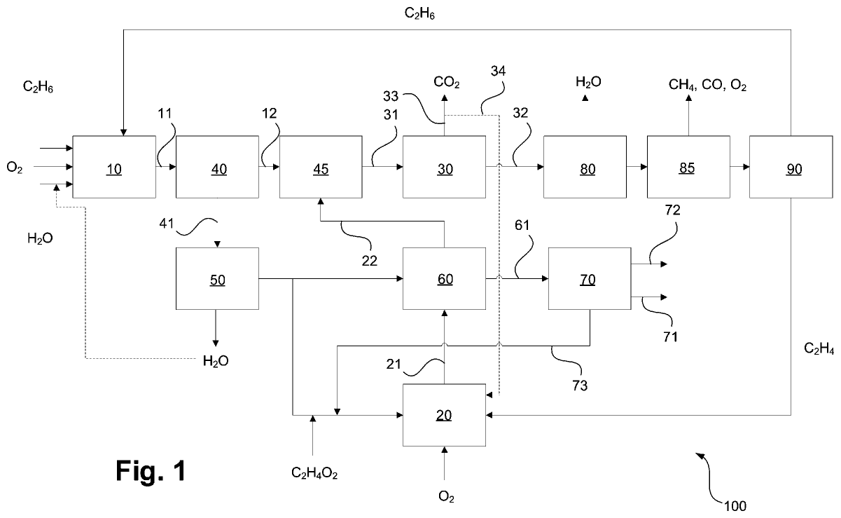

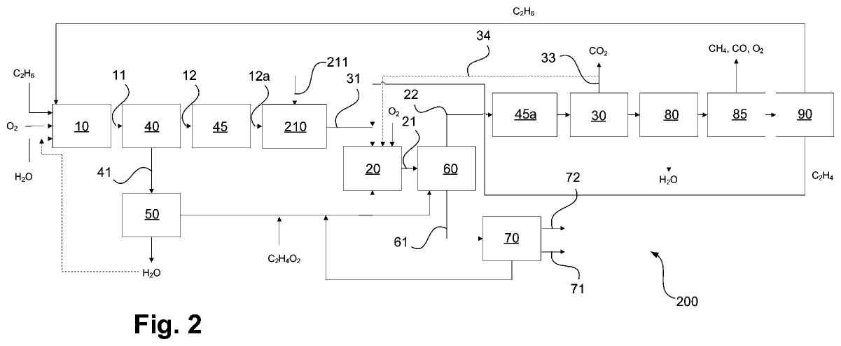

WO 2000/069802 A1 is also cited in the above-mentioned documents, and describes the sequential integration of an ODHE and a vinyl acetate synthesis, in particular with MoVNb-based catalysts for the ODHE, and in particular Pd-based catalysts for the vinyl acetate synthesis. In this document, a carbon dioxide removal is provided either only in the gas stream downstream of the ODHE, i.e., upstream of the vinyl acetate synthesis (FIG. 1 in the document), or the carbon dioxide contained in the gas stream after the ODHE is conveyed in its entirety into the vinyl acetate synthesis, and a carbon dioxide removal takes place only downstream of the vinyl acetate synthesis (FIG. 2). This document also does not mention the presence of methane in the process streams, and thus also does not contain any approach for methane separation in an integrated process. This document does not disclose any solutions for trace removal of, for example, carbon monoxide, acetylene, and/or oxygen. A corresponding intermediate step is explicitly excluded according to the claims. A suitable technical solution for the task of a carbon monoxide removal is therefore also not disclosed. Rather, the document focuses on an embodiment of the ODHE in which no carbon monoxide is produced, which, as explained above, does not correspond to the prior art.

WO 2001/090042 A1 likewise describes an integration of an ODHE and a vinyl acetate synthesis. In particular, Pd-doped catalysts of the MoₐPd_{b}X_{c}Y_{d} type are used for the ODHE. The document only indicates the removal of carbon dioxide, together with “inerts”, only from a return stream of the vinyl acetate synthesis to the ODHE solely in an drawing, without further explanation. In a schematic drawing, a carbon monoxide removal upstream of the vinyl acetate synthesis is outlined, without however demonstrating advantageous solution approaches for this. According to the description, no carbon monoxide, or only an amount of less than 100 ppm, is produced in the ODHE, which does not correspond to the behavior under technically relevant conditions (cf. above). Embodiments regarding acetylene in the product stream of the ODHE and a removal upstream of the vinyl acetate synthesis are not included.

Further publications which also disclose corresponding combinations, but not the measures proposed according to the invention, are, for example, US 2005/0148791 A1, U.S. Pat. No. 5,066,365 A, US 2014/066650 A1 and US 2002/058849 A1.

Irrespective of the aforementioned documents, WO 2019/175732 A1 also describes a linking of an ODHE with a vinyl acetate synthesis using various catalysts (inter alia, MoVTecNbOx-based systems are used). In this case, an oxidation of carbon monoxide and an acetylene removal take place directly in the gas phase fraction of the outlet stream of the ODHE, using at least one metal or a corresponding metal oxide selected from groups 11, 4, 7 or 9, or from the lanthanides or actinides. In particular, Cu, Ag, Au or combinations thereof are explicitly mentioned. However, detailed embodiments only extend to metals of group 11, and the optional use of CeO₂ or ZrO₂ as a promoter. Metals or their oxides from groups 8 (in particular Ru) and 10 (in particular Ni, Pd, Pt), on the other hand, are not named as being suitable for this purpose at any point in the application document. At no point does the application mention the advantageous positioning—in particular with regard to the pressure level—of the claimed oxidation of carbon monoxide. A further integration of the two processes is not disclosed. With regard to carbon dioxide removal, in this document only quite general instructions are found for an “inert removal unit”-without any specific details. However, it does not contain selective carbon dioxide removal, nor does it facilitate a needs-based adjustment of a proportion of a single or all inert components in the feed stream of the vinyl acetate synthesis, in particular it does not facilitate the needs-based adjustment of the carbon dioxide content. Rather, a joint, i.e., non-selective, removal of all inert components in one step is proposed. As is known to those skilled in the art, such a removal is not possible according to the prior art—in particular not by means of absorptive scrubbing. According to specific embodiments, oxygen and acetylene are practically completely absent in the feed stream of the vinyl acetate synthesis.

The removal of carbon dioxide conventionally takes place in particular by means of absorption (e.g., regenerable amine scrubbing and non-regenerable alkali scrubbing). Owing to its strong interaction with suitable solvents or scrubbing liquids, carbon dioxide can likewise be removed comparatively easily from the product mixture, wherein it is also possible to use known processes for removing carbon dioxide, in particular corresponding scrubbing (for example amine scrubbing). The exhausted scrubbing agent in amine scrubbing is then regenerated in a separate column, wherein substantially pure carbon dioxide is released by desorption and is available as a relatively pure stream for further use. A further fine purification and reduction of the carbon dioxide content is possible with an alkali scrubbing. Carbon dioxide can therefore be separated as required and can be obtained virtually as a pure substance in the associated regeneration. Due to the solubility increasing with the pressure, preferably, and in particular also within the scope of the present invention, an absorptive carbon dioxide removal takes place at elevated pressure, i.e., more than 5 bar, in particular more than 10 bar and in particular more than 15 bar.

In the context of an advantageous separation and purification sequence, in the context of the invention, it is also possible in particular to use cryogenic and non-cryogenic distillation processes, wherein a demethanizer and a C2 splitter are used according to the invention.

In a demethanizer, a light(er) fraction (C1 minus fraction) is formed as the top stream, while a heavy (heavier) (C2 plus fraction) is obtained as the bottom stream. The C1 minus fraction is in particular enriched in methane and/or carbon monoxide, while the bottom fraction is depleted of these components or is free of these components. Here, the term “enriched” is intended to denote any increased content, but in particular a content increased by at least the factor 2, 5, 10, 100 or more, for one or more components, based on a starting mixture before the enrichment. Accordingly, the term “depleted” is used to denote any reduced content, but in particular a content reduced by at least the factor 2, 5, 10, 100 or more, for one or more components, based on a starting mixture before the depletion.

In the case of an ODHE, the bottom fraction is thus predominantly or exclusively ethane and ethylene. A demethanizer is typically operated, and in particular also within the scope of the present invention, in a pressure range between 10 bar and 50 bar, preferably 15 bar and 40 bar, particularly preferably between 20 bar and 35 bar. In particular, the top temperature must be correspondingly low to ensure a liquid-phase return. Typical temperatures in this case are below −50° C., in particular below −70° C., and further in particular below −80° C.

EP 3 339 277 A1 discloses statements regarding demethanizers in an ODHE process. EP 3 456 703 A1, for example, describes an ODHE process which initially includes a demethanizer in the decomposition section. The top stream formed here, comprising in particular methane, is supplied to a pressure swing adsorption (PSA) in order to recover ethylene contained in this stream and return it to the decomposition section of the ODHE system.

Furthermore, the separation of ethylene and unreacted ethane in a so-called C2 splitter is required. Ethylene is typically obtained at a purity of more than 98%, preferably more than 99%, particularly preferably more than 99.5%. Ethane is recirculated, both in steam cracking and in ODHE processes, as reaction input, i.e., for example, back to the ODHE reactor. In order to minimize a loss of ethylene here, this fraction is usually also obtained in high purity. A C2 splitter is typically operated, and in particular also within the scope of the invention, in a pressure range between 5 bar and 50 bar, preferably 7 bar and 40 bar, particularly preferably between 8 bar and 35 bar. In addition-depending on the pressure used-a relatively low top temperature is required, i.e., typically less than −5° C., in particular less than −20° C., and more particularly less than −50° C.

Further separation and purification steps which are known and can also be used in the context of the invention include (as a non-exhaustive list) phase separations, in particular by means of separators and decanters, reactive, in particular catalytic, removal of oxygen and trace compounds (in particular carbon monoxide and acetylene), in particular with the addition of hydrogen and/or oxygen, adsorptive cleaning and drying processes, which can use regenerable and non-regenerable adsorbents depending on the material and intended use, and pressure swing adsorption (PSA) and membrane processes.

An object of the invention can in particular be specified, in a first aspect, in the creation of a process control which enables a particularly efficient integration of the ODHE and vinyl acetate synthesis, and thus the use of the product mixture of the ODHE for the vinyl acetate synthesis.

The prior art provides the point of departure as follows: In addition to ethylene, a “conventional” ODHE provides in particular acetic acid as a by-product. The ratio of ethylene and acetic acid can be adjusted within certain limits as required.

At the outlet of the ODHE reactor, a minimum oxygen content must usually be ensured in order to prevent catalyst damage. However, this residual oxygen content is minimized according to the prior art, and is usually at least 0.41 mol %, but not more than 1.5 mol %, less than 0.8 mol % or less than 0.6 mol % in the entire outlet stream of the ODHE (i.e., in the humidified process gas stream before the condensate removal). Thereafter, measures can be provided for an oxygen removal, as described, for example, in the documents already mentioned above (e.g. US 2010/0256432 A1).

Ethylene is usually provided as a pure product, and correspondingly separated from other hydrocarbons, in particular ethane and methane, with a complicated procedure. Methane can in particular be introduced as part of the ODHE feed stream.

The vinyl acetate synthesis requires ethylene and acetic acid as a reaction input, typically in a ratio of from 3 to 1 to 1 to 1.

On the other hand, oxygen must be added as a reactant for the vinyl acetate synthesis.

However, for further integration of the two processes, it is particularly important to take into account the following aspects, for which the invention, especially in its embodiments, provide advantageous solutions:
In addition to ethylene and acetic acid, further by-products are also formed in the ODHE—in addition to carbon dioxide, in particular acetylene and carbon monoxide—which must be removed by suitable process steps. In particular acetylene and carbon monoxide have a negative effect on subsequent process steps—in this case also on the vinyl acetate synthesis.
In the vinyl acetate synthesis, a suitable diluent is usually added, which in turn has to be removed from the product mixture of the vinyl acetate synthesis. Carbon dioxide is often used for this task, and further carbon dioxide is additionally produced as a by-product in the vinyl acetate synthesis. Accordingly, the product stream from the vinyl acetate synthesis must also be subjected to a carbon dioxide purification. However, the inlet stream of the vinyl acetate synthesis can also in particular contain no or only small amounts of carbon dioxide.
In particular, process-optimized process control of carbon dioxide is not yet part of available technologies or developments. Instead, carbon dioxide is typically removed by isolated purification steps, and is not utilized within an integrated overall process. In particular, the compression of gas streams requires a lot of effort with regard to the required complex apparatuses (compressors, often multi-stage) and operating costs (energy consumption for the compression). A sequential connection of independent ODHE- and vinyl acetate synthesis processes requires repeated compression and expansion in the process chain. However, known approaches for process integration of the ODHE and vinyl acetate synthesis do not have an optimized approach for selecting suitable process controls and optimized pressure levels.

As mentioned, a carboxylic acid is formed in the ODH as a secondary or by-product; when ethane is used as an input into the ODH (i.e., in ODHE), acetic acid is formed. An adjustment of the ratio of ethylene to acetic acid is possible by suitable measures, as described in WO 2018/115418 A1, WO 2018/115416 A1 and WO 2019/243480 A1, such as the choice of the catalyst or the reaction conditions, in particular adjusting the partial pressure of water in the process gas stream of the ODHE (cf. in particular WO 2018/115416 A1).

In the context of the invention, the aim is in particular to have a ratio of ethylene to acetic acid between 1:1 and 10:1, in particular between 2:1 and 5:1 and further in particular between 2.5:1 and 4:1. This acetic acid, together with reaction water, can be separated off comparatively easily from a corresponding product mixture of the oxidative dehydrogenation by condensation and/or a water scrubbing. The product stream is therefore separated into a condensate phase and a gas phase.

By suitable processes known to a person skilled in the art, if necessary, enrichment of the acetic acid from the condensate phase and recovery as its own target product is then also possible, which can be supplied to the vinyl acetate synthesis. The gas phase from the product stream of the ODHE then contains, in particular, ethylene, but also further constituents, such as unreacted ethane, methane (in particular from the feed of the ODHE), and in particular carbon monoxide and carbon dioxide, and small amounts of acetylene (less than about 300 to 400 ppmv) as by-products.

In the context of the invention, the oxidative dehydrogenation is advantageously carried out at a pressure level of 1 bar to 10 bar, in particular 2 bar to 6 bar- and the vinyl acetate production advantageously at a pressure level of 2 bar to 50 bar, in particular 5 bar to 15 bar.

Building on these principles, the invention discloses in a primary aspect an optimized solution for efficient carbon dioxide removal from an integrated ODHE and vinyl acetate synthesis. This makes it possible, in particular, in a simple manner to adjust the carbon dioxide content in the inlet stream of the vinyl acetate synthesis in accordance with requirements. The invention skillfully makes use of the following facts:
1. In the vinyl acetate synthesis, a certain proportion of carbon dioxide is used in the reaction feed of the vinyl acetate synthesis as a diluent, and carbon dioxide can thus run through the vinyl acetate synthesis, and/or is even desired here in certain proportions. The carbon dioxide content in the feed to the vinyl acetate synthesis can be 0 to 30%, in particular 0 to 15%, more particularly 0 to 5%.
2. In the vinyl acetate synthesis, in any case, carbon dioxide is produced as a by-product.
3. Owing to its strong interaction with suitable solvents or scrubbing liquids, carbon dioxide can likewise be removed comparatively easily from the product mixture, wherein it is possible to use known processes for removing the carbon dioxide, in particular corresponding scrubbing (for example amine scrubbing). Carbon dioxide can therefore be separated as required and can virtually be obtained as a pure substance in the associated regeneration. Such a carbon dioxide removal can take place at a suitable point in the process. A further fine purification and reduction of the carbon dioxide content is possible, as already mentioned above, in principle by a downstream alkali scrubbing.

In addition, the invention discloses, in particular in corresponding advantageous embodiments, an advantageous selection of the pressure levels of the corresponding process steps. As explained below, further necessary process steps, such as a demethanizer and/or C2 splitters, are also advantageously included and taken into account as needed. In principle, reaction and process steps can also be arranged within the overall process at a suitable point in order to remove by-products or trace compounds. In particular, the process according to the invention can contain necessary purification steps for removing traces, in particular of carbon monoxide, acetylene and/or oxygen.

In a corresponding group of embodiments, and/or a further aspect, which is/are explained further below, the invention therefore addresses the problem that the product stream of the ODHE, as mentioned above, contains, in addition to ethylene and components, such as ethane and carbon dioxide, in particular also carbon monoxide and acetylene (typically in a content of less than 300 to 400 ppmv). This relates in particular to a gas phase of the product stream of an ODHE after condensation.

The aforementioned object of the invention is achieved in that the corresponding gas phase product streams of an ODHE and a vinyl acetate synthesis are at least partially subjected to a single carbon dioxide removal. This carbon dioxide removal is preferably designed as an absorptive and regenerative scrubbing. In order to realize the most efficient possible carbon dioxide removal, an increased pressure is advantageously selected for the carbon dioxide removal. In the context of the present invention, the carbon dioxide removal is therefore preferably carried out at a higher pressure level than the ODHE and the vinyl acetate synthesis. The corresponding gas phase product streams of these steps (i.e., the ODHE and the vinyl acetate synthesis) can therefore be supplied to a common compression, and then to the common carbon dioxide removal according to the invention. The common compression can be configured in one or more stages. Accordingly, the two partial streams can also only be combined after a suitable compressor stage. That is, in one or more first compressor stages, only the product stream of one of the two steps is compressed, and then the further joint compression takes place in one or more second compressor stages.

When the scrubbing agent is regenerated, the carbon dioxide removed in an absorptive scrubbing can then be obtained again as a carbon dioxide fraction and supplied to the feed stream of the vinyl acetate synthesis in suitable proportions in order to thereby enable a dilution with carbon dioxide according to requirements. A particular advantage here is that the carbon dioxide concentration at the inlet of the vinyl acetate synthesis can be set independently of the carbon dioxide content in the product stream of the ODHE, in a very wide range. This embodiment therefore represents a particularly advantageous variant.

In principle, the supply of a substream of the product mixture of the ODHE into the vinyl acetate synthesis can also take place upstream of the carbon dioxide removal. Such a process control is basically known from WO 2018/114747 A1. However, since such a substream has a certain composition in particular with regard to carbon dioxide and ethane and optionally methane (cf. explanations below) and additionally also contains carbon monoxide and acetylene, which are known poisons for catalysts for the vinyl acetate synthesis, such a process control does not appear advantageous, or only appears possible for a small proportion in relation to the total feed stream of the vinyl acetate synthesis. Furthermore, fluctuations of the process gas composition as part of fluctuations in the operation of the ODHE and/or the vinyl acetate synthesis are to be expected, whereby in turn a further undesired feedback to the vinyl acetate synthesis is to be expected.

In order to achieve the stated aims, the invention proposes a method for the production of vinyl acetate in which ethane and oxygen are subjected to an oxidative catalytic dehydrogenation (ODHE) in one or more first process steps, to obtain a first product mixture containing ethane, ethylene, water, acetic acid, and carbon dioxide, and in which, in one or more second process steps, ethylene and acetic acid are subjected to a catalytic vinyl acetate synthesis, to obtain a gaseous second product mixture containing ethylene, vinyl acetate, and carbon dioxide, wherein at least a portion of the ethylene and at least a portion of the acetic acid which are subjected to the catalytic reaction in the one or more second process steps are formed from at least a portion of the ethylene and at least a portion of the acetic acid from the first product mixture.

According to the invention, it is provided that, as already stated above using other words, a first subsequent mixture containing ethane, ethylene and carbon dioxide is formed from at least a portion of a first product mixture, and that a second subsequent mixture containing ethylene and carbon dioxide is formed using at least a portion of the second product mixture. In this case, the formation of the first subsequent mixture includes subjecting at least a portion of the first product mixture and the first subsequent mixture to a condensing deposition of water and acetic acid, producing a condensate fraction containing water and acetic acid, wherein at least a portion of the acetic acid which is subjected to the catalytic vinyl acetate synthesis in the one or more second process steps is formed by at least a portion of the acetic acid from the condensate fraction.

It is further provided that, using at least one portion of the first subsequent mixture and using at least one portion of the second subsequent mixture, more precisely by combining at least most of the first subsequent mixture and the second subsequent mixture, a third subsequent mixture containing ethane, ethylene and carbon dioxide is formed, wherein the formation of the second subsequent mixture comprises subjecting at least a portion of the second product mixture and of the second subsequent mixture to a vinyl acetate separation to obtain a precursor fraction containing vinyl acetate.

It is also provided that at least a portion of the third subsequent mixture is subjected to one or more third process steps, wherein the one or more third process steps comprise a common carbon dioxide removal, wherein a fourth subsequent mixture containing ethane and ethylene, and a carbon dioxide fraction, are formed in the one or more third process steps, and wherein at least a portion of the fourth subsequent mixture is subjected to a separation in which an ethane fraction and an ethylene fraction are formed.

At least a portion of the ethylene, which is subjected to the catalytic vinyl acetate synthesis in the one or more second process steps, is formed by at least a portion of the ethylene from the ethylene fraction, and/or at least a portion of the ethane, which is subjected in the one or more first process steps to the oxidative catalytic dehydrogenation, is formed by at least a portion of the ethane from the ethane fraction.

Further details and advantages of the measures proposed according to the invention have already been explained in detail. An input stream into the vinyl acetate synthesis can in particular have a content between 0 and 30% carbon dioxide, and the or at least one of the third process steps can be carried out in a pressure range which is above and in particular at least 1 bar, 2 bar, 5 bar or 10 bar above a pressure range in which the first and second process steps are carried out.

In principle, the corresponding product stream can be supplied directly to the vinyl acetate synthesis (i.e., the one or more second process steps) after the common carbon dioxide removal according to the invention (i.e., the fourth subsequent mixture downstream of the third process step or a corresponding portion thereof). In particular, ethane and/or methane still present act as inert diluents in the vinyl acetate synthesis.

However, in practice, proportions of carbon monoxide and/or acetylene still contained in the gas stream are contrary to such an embodiment, and can act as catalyst poisons in the vinyl acetate synthesis if they have not been removed beforehand. A further purification, as described further below, is therefore advantageous as part of an integrated process. This can take place upstream or downstream of the carbon dioxide removal, i.e., upstream or downstream of the third process step, wherein in the first case, which is the case according to the invention, the formation of the first subsequent mixture comprises the at least partial removal of carbon monoxide and/or acetylene, and in the latter case the fourth subsequent mixture is subjected to an at least partial removal of carbon monoxide and/or acetylene.

For the additional separation steps mentioned below, however, in accordance with the temperature levels selected, a drying is optionally also upstream of the carbon dioxide removal according to the invention, in order to avoid distortions due to freezing of water and/or carbon dioxide.

As already explained in other words, the carbon dioxide removal advantageously comprises a regenerative adsorptive carbon dioxide removal in which a scrubbing liquid is used which is loaded with carbon dioxide in the regenerative absorptive carbon dioxide removal, and from which the carbon dioxide is expelled to obtain the carbon dioxide fraction. At least a portion of the carbon dioxide fraction can be passed as a diluent through the one or more second process steps, i.e., the vinyl acetate synthesis, as already explained.

As mentioned, the formation of the first subsequent mixture comprises subjecting at least a portion of the first product mixture and the first subsequent mixture to a condensing deposition of water and acetic acid (hereinafter also “condensate separation”) to obtain a (liquid) condensate fraction containing water and acetic acid. At least a portion of the acetic acid, which is subjected to the catalytic vinyl acetate synthesis in the one or more second process steps, is, as likewise mentioned, formed by at least a portion of the acetic acid from the condensate fraction. The latter can in particular be formed in a condensate removal (also referred to as acetic acid purification) which is downstream of the condensate separation. Reference can also be made to the above explanations regarding this aspect of an embodiment of the invention.

In one embodiment of the invention, the formation of the third subsequent mixture can comprise subjecting at least a portion of the first subsequent mixture and at least a portion of the second subsequent mixture to a common compression. As already explained in this respect, a multistage compressor of known type can be used, for example, to which at least a portion of the first subsequent mixture can be supplied upstream of a first compressor stage and at least a portion of the second subsequent mixture can be supplied upstream of a second compressor stage, wherein the second compressor stage can be arranged upstream or downstream of the first compressor stage.

In the process proposed according to the invention, the formation of the second subsequent mixture comprises subjecting at least a portion of the second product mixture and of the second subsequent mixture to a vinyl acetate separation, as already mentioned, to obtain a precursor fraction containing vinyl acetate. In the vinyl acetate separation, a portion of the acetic acid can be used as absorption medium from the mentioned condensate fraction.

At least a portion of the precursor fraction can be subjected in the scope of the invention to a vinyl acetate purification in which a vinyl acetate fraction, a by-product fraction, and an acetic acid fraction can be formed. In this case, in particular at least a portion of the acetic acid which is subjected to the catalytic vinyl acetate synthesis in the one or more second process steps can be formed by at least a portion of the acetic acid from the acetic acid fraction from the vinyl acetate purification.

As already mentioned, at least a portion of the fourth subsequent mixture is subjected to a separation in which an ethane fraction and an ethylene fraction are formed. The separation comprises in particular a drying of a type known per se and described above, followed by a demethanization according to the invention, and a C2 separation in a C2 splitter. However, other separation sequences and separation arrangements can also be used. For further details, reference is made to the explanations above. At least a portion of the ethylene which is subjected to the catalytic vinyl acetate synthesis in the one or more second process steps is then formed by at least a portion of the ethylene from the ethylene fraction, and/or at least a portion of the ethane which is subjected to the oxidative catalytic dehydrogenation in the one or more first process steps is formed by at least a portion of the ethane from the ethane fraction. Further details on this aspect have also been explained previously.

In a preferred embodiment of the invention, a C2 splitter can be integrated into the process, and in particular the feed stream of the vinyl acetate synthesis can be depleted of ethane or ethane can be quantitatively removed from this stream. In particular, an embodiment in which the ethylene fraction from the C2 splitter still contains a certain proportion of ethane is particularly advantageous in order to minimize the effort (apparatus/investment costs/operating costs) of this separation. In particular, the ethane content in the stream for the vinyl acetate synthesis, i.e., the ethylene fraction which in this case is conveyed into the one or more second process steps for the vinyl acetate synthesis without further separation processing, can in this case be between 0 and 20 mol %, in particular between 0.01 and 10 mol %, more particularly between 0.5 and 5 mol % ethane. A gas phase from the vinyl acetate synthesis, which correspondingly contains ethane and unreacted ethylene, can also be returned at a suitable location upstream of the C2 splitter.

Due to the joint carbon dioxide removal according to the invention, a certain concentration of ethane can build up in the circulation via the vinyl acetate synthesis due to the recirculation, but the C2 splitter provided according to the explained embodiment causes a continuous discharge of ethane and avoids excessive ethane dilution. An optimal ethane dilution in this stream can also be achieved in this way. The ethane stream of the C2 splitter (the ethane fraction explained) is advantageously at least partially returned to the ODHE and, on the other hand, preferably contains the lowest possible fraction of ethylene; however, in this case as well, quantitative removal of ethylene is not absolutely necessary. The ethylene content can be, for example, between 0 and 20 mol %, in particular between 0.01 and 10 mol %, more particularly between 0.5 and 5 mol %.

Within the scope of the invention, a C2 splitter can advantageously already be operated at a relatively low pressure of about 5 bar (see the explanations above). For technical implementation, a pressure that is slightly above that of the vinyl acetate synthesis is particularly advantageous. “Slightly above” is understood here to mean a pressure difference of up to 15 bar, preferably of up to 10 bar, and particularly preferably of up to 5 bar.

The demethanizer provided in one embodiment of the invention can, in particular, serve or be configured for at least partial removal in particular of methane, but in particular also of carbon monoxide, from the process gas stream, i.e., from the fourth subsequent mixture or from a further subsequent mixture thereof, in order to avoid or limit an enrichment in a circulation via the vinyl acetate synthesis, for example. The demethanizer can be arranged at a suitable location of the process, and in particular can be connected upstream of a C2 splitter or downstream in the top stream of a C2 splitter. In addition to methane, the top stream of a demethanizer can also contain, in particular, carbon monoxide and oxygen, provided that these are not removed at an upstream trace removal—e.g. in a crude gas treatment explained below. As described above, ensuring a liquid return (condensation of the C1 minus fraction) requires correspondingly low temperatures. Nevertheless, a demethanizer can also be operated advantageously in the context of the present invention—if a correspondingly low head temperature is set—at a pressure that is slightly above the vinyl acetate synthesis.

In a particularly advantageous embodiment of the invention, on the other hand, a complete or at least predominant removal of carbon monoxide takes place upstream of the demethanizer in a crude gas treatment referred to elsewhere as trace removal, and/or in process steps of any type, and no complete separation of methane from the process stream in the demethanizer is required. Rather, a depletion is sufficient, i.e., a reduction of the mass of methane contained in the process stream by at least 90%, preferably at least 50% and particularly preferably at least 25%. After the vinyl acetate synthesis, the remaining methane content can be separated comparatively easily from the product stream of the vinyl acetate synthesis. It is thus essential to reduce the amount of methane upstream of the vinyl acetate synthesis to a level suitable for the vinyl acetate synthesis, and/or to avoid an enrichment of methane in a possible circulation. In contrast to the usually desired provision of a pure ethylene product, however, a quantitative or virtually quantitative removal of methane from the ethylene feed of the vinyl acetate synthesis is not required. This represents a further particular advantage of the invention over conventional separation and purification sequences. This particular advantage is made possible only by the upstream carbon monoxide removal (crude gas treatment).

Aspects of embodiments of the invention, which can be combined in any desired manner with all explained embodiments and with one another, thus comprise in particular the idea that at least methane and/or carbon monoxide are at least partially removed from the process stream by means of a demethanizer, and/or that the carbon dioxide removal takes place at a higher pressure level than the ODHE and the vinyl acetate synthesis, and wherein a single or multistage compression takes place upstream of the carbon dioxide removal. Such aspects further comprise in particular an additional removal of traces, in particular of carbon monoxide and/or acetylene. The trace removal can be provided in any desired manner. A removal of carbon monoxide and/or acetylene can take place before or after a compression and before a carbon dioxide removal. In particular, in all embodiments of the process according to the invention, the outlet stream from an ODHE reactor (moist process gas, i.e., the first product mixture) can contain more than 0.40 mol. %, in particular more than 0.6 mol. %, or more than 1.5 mol. % oxygen. After a removal of carbon monoxide, the oxygen content can in particular be more than 0.01 mol. %, more than 0.05 mol. %, or more than 0.20 mol. %, but in particular always less than 0.4 mol. %. The oxidative dehydrogenation can be operated in particular in such a way that ethylene and acetic acid are produced in the first product mixture in a ratio between 1 to 1 and 10 to 1, in particular in a ratio of 2 to 1 to 5 to 1 and more particularly in a ratio of 2.5 to 1 to 4 to 1.

In a group of configurations which has already been discussed above and is now explained further, the invention therefore addresses the problem that the product stream of the ODHE, as mentioned above, contains, in addition to ethylene and components, such as ethane and carbon dioxide, in particular also carbon monoxide and acetylene (typically in a content of less than 300 to 400 ppmv). This relates in particular to a gas phase of the product stream of the ODHE, i.e., of the first product mixture, after condensation.

As mentioned, in particular acetylene and carbon monoxide must be removed as completely as possible from the feed stream of the vinyl acetate synthesis, since both are potential catalyst poisons for the vinyl acetate synthesis. Existing approaches do not demonstrate an advantageous solution for removing these traces as part of an integrated process. In embodiments, the invention thus discloses an optimized solution for a trace removal (in particular of acetylene and/or carbon monoxide) in an integrated arrangement of the ODHE and the vinyl acetate synthesis. In a first embodiment (with a “crude gas treatment”, see above), the invention makes use of the following facts:
1. An oxidative removal of acetylene and possibly carbon monoxide in the gas phase of the outlet stream of an ODHE is known.
2. The ODHE requires a residual oxygen content at the reactor outlet. The gas phase of the outlet stream of the ODHE thus contains a certain amount of oxygen.
3. At the same time, oxygen is added for the vinyl acetate synthesis as described above.

A single catalytic conversion of oxygen and acetylene in the gas phase of an outlet stream from an ODHE via suitable catalysts in such a crude gas treatment is described, for example, in WO 2020/187572 A1 and WO 2018/153831 A1. However, therein, the aim is to virtually quantitatively remove both oxygen and acetylene in order to achieve the desired ethylene quality in the further product purification, in particular by fractionation without additional measures, and, on the other hand, to avoid the accumulation of oxygen in certain fractions, in particular in a C1 minus fraction. Carbon monoxide can also be at least partially converted in this case.

If an ODHE and a vinyl acetate synthesis are integrated, it is optionally possible, according to an embodiment not forming part of the invention, to also not provide for a complete oxygen removal here, before the product mixture of the ODHE (also referred to above as “first product mixture”) is supplied as a source for ethylene and acetic acid to the vinyl acetate synthesis. Residual oxygen can then be transferred to the vinyl acetate synthesis. It serves there as a reactant. An increased oxygen content at the outlet of the ODHE can thus be permitted at first, which is particularly advantageous for the operation of the ODHE, which requires a minimum oxygen content in the outlet stream.

As a result of an increased oxygen content, in particular a higher tolerance of the ODHE for any operating fluctuations and an overall longer catalyst service life can be achieved. If necessary, an additional feed of oxygen and/or hydrogen can also take place before or during the crude gas treatment (see WO 2020/187572 A1). In this case, the oxygen content at the input of the crude gas treatment is now advantageously also an additional degree of freedom. In addition to the oxygen from the outlet of the ODHE, further oxygen can thus be fed in as required before the crude gas treatment, and an optimal performance of this crude gas treatment can thus be achieved for acetylene and/or carbon monoxide removal. Accordingly, the further feed of oxygen in the feed of the vinyl acetate synthesis can be reduced. In principle, catalysts known to those skilled in the art can be used in the crude gas treatment.

In particular, the catalysts described in WO 2020/187572 A1 and WO 2018/153831 A1 are used. Therefore, reference is accordingly made at this point to these two documents and the further statements contained therein. In particular, catalysts containing copper oxide, catalysts containing at least one of the elements copper, manganese, zinc, nickel, platinum, palladium, rhodium, and/or ruthenium can be used in the crude gas treatment. According to the invention, such a crude gas treatment can be arranged before or after a compression of the gas phase of the outlet stream of the ODHE, or also at a suitable pressure level between two compressor stages. In other words, the component mixture referred to above as the “first product mixture” is supplied to the crude gas treatment.

Ethane and possibly methane contained in the gas phase of the ODHE can also be fed into the vinyl acetate synthesis without separation and serve there as an inert and/or diluent medium. In embodiments, further process steps can then be included which, as explained further below, can comprise, in a suitable arrangement, in particular a drying, an optional demethanizer and/or a C2 splitter, in addition to a carbon dioxide removal, as described in detail above.

Unconverted ethylene can then be recycled completely or partially to the vinyl acetate synthesis. In addition to ethylene, other components, in particular carbon dioxide, ethane, and/or methane, can also be contained in this residual gas stream. If the crude gas treatment does not achieve sufficient carbon monoxide removal, or is operated in such a way that only acetylene is sufficiently removed, remaining carbon monoxide can be removed in the process stream after the crude gas treatment-if necessary in a demethanizer, which also functions for at least partially removing methane. If the residual gas stream from the vinyl acetate synthesis does not contain any proportions of, in particular, acetylene and/or possibly carbon monoxide, the recirculation described above can take place without involving the crude gas treatment.

If, in contrast, the residual gas stream from the vinyl acetate synthesis also contains fractions of in particular acetylene and/or possibly carbon monoxide, in one embodiment of the invention, however, the crude gas treatment can also advantageously be included in the recirculation noted above. For this purpose, the residual gas stream is at least partially fed from the vinyl acetate synthesis into the gas phase of the outlet stream of the ODHE. In this way, the residual gas stream from a vinyl acetate synthesis, after removal of condensable fractions, is then also subjected to a common crude gas treatment. No additional purification stages are thus required in the residual gas stream of the vinyl acetate synthesis, and in these cases the crude gas treatment can also be arranged before or after a compression of the gas phase of the outlet stream of the ODHE, or also at a suitable pressure level between two compressor stages. An at least partial feed of the residual gas stream from the vinyl acetate synthesis takes place at a suitable point upstream of the crude gas treatment.

In a further embodiment (“tail-end hydrogenation”), the invention makes use of the following facts:
1. The removal of acetylene from ethylene-rich process streams via a so-called tail-end hydrogenation is known.
2. A removal of carbon monoxide can take place in a demethanizer above the C1 minus fraction.
3. An oxygen removal is also possible via the C1 minus fraction in a demethanizer.
4. Alternatively, an independent oxidative conversion of CO with oxygen is also known.

Then, the gas phase of the outlet stream of an ODHE and the residual gas stream of the vinyl acetate synthesis are initially combined at least in part. The combined gas mixture then goes through suitable process steps, which—as already noted for the crude gas treatment and explained in detail below—can include, in a suitable arrangement, in particular a carbon dioxide removal, drying, a demethanizer and/or a C2 splitter. In particular, the demethanizer now serves to remove carbon monoxide contained in the combined gas mixture. At the same time, fractions of methane and/or oxygen are removed. In this embodiment, the removal of carbon monoxide and carbon dioxide is advantageous, since these two compounds have a negative influence on the activity and/or selectivity in the subsequent tail-end hydrogenation. According to the invention, acetylene is then removed in a downstream tail-end hydrogenation, which can alternatively either be located upstream or downstream of a C2 splitter. In this way, traces of carbon monoxide and/or acetylene can thus likewise ultimately be removed in an optimized process chain, and in turn no additional purification stages are necessary in the residual gas stream of the VAM synthesis for this task. In the tail-end hydrogenation, catalysts known in principle to those skilled in the art can be used. In particular, catalysts containing at least one of the elements Ni, Pd, Rh, Ir, Ru, Pt, Ce, Ag and Au can be used.

As regards the features and advantages of the system proposed according to the invention, and advantageous embodiments thereof, reference is made explicitly to the above explanations regarding the process according to the invention and its embodiments. In particular, the system proposed according to the invention and/or corresponding embodiments are configured to carry out corresponding process variants.

The invention is further explained below with reference to examples corresponding to embodiments of the invention, and associated figures.

Where reference is made below to aspects of a process, these explanations relate analogously to corresponding systems and their embodiments. The same applies to process steps and system components. Process steps and system components of the same or comparable function and/or technical implementation are indicated by the same reference signs.

The process illustrated in FIG. 1 is designated as a whole by 100. In the process 100, the required reactants ethane C₂H₆ and oxygen O₂ are supplied to an ODHE (previously referred to as the one or more first process steps and/or process components 10). Depending on the ethane source and any upstream purification steps, the ethane stream C₂H₆ may also contain certain fractions, for example of methane and/or higher hydrocarbons. In addition, water vapor H₂O may be added as a diluent if needed. The water required for this can in particular also originate at least partially from the raffinate water phase of an optional acetic acid purification and/or condensate separation 50, as illustrated by a dashed arrow.

In the ODHE, i.e., in the one or more first process steps and/or process components 10, a product mixture is formed, which was previously referred to as the first product mixture and is indicated here as 11. After cooling and condensation (not shown), a phase separation or deposition 40 of a condensate fraction 41 occurs—i.e., an aqueous phase, which can contain acetic acid. This can be fed to the aforementioned acetic acid purification or condensate separation 50. The remaining gas phase, which substantially contains unreacted ethane, as well as ethylene and carbon dioxide, and certain fractions of carbon monoxide and optionally further trace compounds-previously also referred to as the first subsequent mixture—is then compressed to a required pressure level in a compression 45 and fed to a carbon dioxide removal, which in this case can be part of one or more process steps and/or corresponding system components which have been referred to above as third process steps and/or system components 30.

In this case, there is a return stream 22 feed from a vinyl acetate synthesis, which here can be part of one or more process steps and/or corresponding system components, which have previously been referred to as second process steps or system components 20, and a subsequent scrubbing 60 corresponding to the pressure level before the compression 45 and/or upstream of a corresponding compressor stage. The return stream 22 was previously also referred to as the second subsequent mixture.

According to the embodiment illustrated here, both the gaseous portion of the ODHE product (first subsequent mixture 12) and the gaseous residual stream from the vinyl acetate synthesis (second subsequent mixture 22) are subjected to a common carbon dioxide removal in the third process step(s) and/or system components 30. A stream that is accordingly introduced at this point, resulting from the combination of the first and second subsequent mixtures (12 and 22), was also referred to above as a third subsequent mixture 31. The carbon dioxide removal in this case comprises in particular an absorptive scrubbing. A carbon dioxide fraction 33 obtained during the regeneration of the loaded absorbent (not explicitly shown) can—if desired—be supplied in part to the vinyl acetate synthesis as a diluent in the form of a material stream 34, in a quantity that is as appropriate and can be easily adjusted at all times. Depending on the requirements, the carbon dioxide removal can also comprise an optional alkali scrubbing (not explicitly shown), which serves to further reduce the carbon dioxide content in the remaining gas stream and/or fourth subsequent mixture 32 with a carbon dioxide content already reduced in the absorptive scrubbing.

After a drying 80, this material stream 32, also referred to above as the fourth subsequent mixture, is supplied further to distillative process steps and a further separation. These distillative process steps include in particular a C2 splitter or a C2 separation 90, which, in particular, divides the process stream into a fraction (“ethane fraction” for short) that is at least enriched for ethane C₂H₆ and also into a fraction (“ethylene fraction”) that is at least enriched for ethylene C₂H₄. Furthermore, however, an optional demethanizer 85 can also be contained in the process arrangement. This optional demethanizer 85 serves to remove light components as the C1 minus fraction, which in particular can contain the components methane, carbon monoxide, and oxygen. The optional demethanizer 85 can optionally be arranged, as shown in FIG. 1, upstream of the C2 splitter 90, or downstream of a corresponding C2 splitter in the ethylene-enriched fraction/ethylene fraction C₂H₄. The ethane-enriched fraction/ethane fraction C₂H₆ in turn, is fed to the ODHE or to the one or more first process steps 10 as a feed stream. The ethylene-enriched fraction/ethylene fraction C₂H₄ is supplied to the vinyl acetate synthesis/to the one or more second process steps 20 as a feed stream. Optionally, a substream of this fraction C₂H₄ can also be diverted and used as a reactant stream for other processes (not explicitly shown).

Further feed streams of the vinyl acetate synthesis/the one or more second process steps 20 comprise in particular a supply of oxygen O₂, a supply of acetic acid, in particular from the aforementioned acetic acid purification and/or condensate separation 50, and the already mentioned provision as required of carbon dioxide from the carbon dioxide removal 30 of from the one or more third process steps. In addition, an acetic acid fraction from a vinyl acetate preparation 70 can be returned, in particular via distillation processes, in the form of a material stream 73, or optionally also from an additional external source, as indicated by C₂H₄O₂.

The outlet stream from the vinyl acetate synthesis or the one or more second process steps 20 is then subjected to the already described scrubbing 60 which, on the one hand, generates a gas stream which, as described, is supplied as a second subsequent mixture 22 to the compression 45 and which contains, in particular, unreacted ethylene and carbon dioxide. Additional carbon dioxide is created as a by-product of the vinyl acetate synthesis. There may be further volatile constituents as well, e.g., oxygen or constituents from the ethylene-enriched fraction or ethylene fraction C₂H₄ from the C2 splitter, in particular ethane. The scrubbing 60 is likewise supplied with an acetic acid stream as a scrubbing agent, which acetic acid stream can originate either from the acetic acid purification or condensate separation 50 and/or the already mentioned, in particular distillative, vinyl acetate purification 70 and/or an, in particular additional, external source. This scrubbing agent is then used to remove the reaction product vinyl acetate from the (second) product mixture of the vinyl acetate synthesis or the one or more second process steps 20, which is then fed to the already mentioned vinyl acetate purification 70, in particular by distillation. In process step 70, the desired end product vinyl acetate is then obtained in the required purity in the form of a vinyl acetate fraction 71. A by-product fraction 72 also precipitates, which in particular contains water, as well as other by-products of the vinyl acetate synthesis.

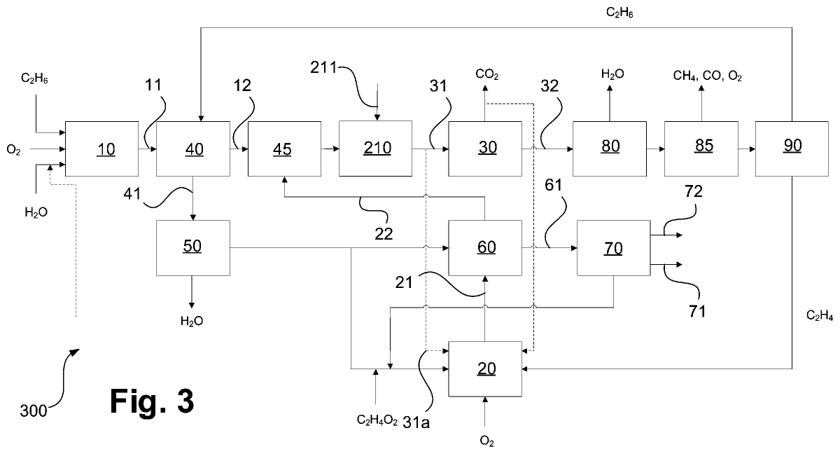

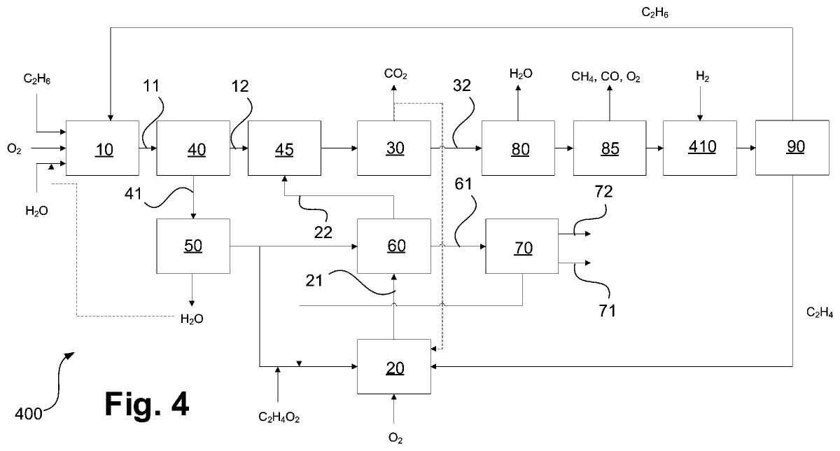

The process 100 can additionally contain further optional purification steps for removing trace compounds, and optional purge streams in order to avoid the accumulation of unwanted trace compounds in return streams. These are not explicitly shown in FIG. 1. Reference is now made to FIGS. 3 and 4.

In FIG. 2, a further process and/or a further system is shown in the form of a schematic flowchart, and is designated as a whole by 200. The explanations for FIG. 1 generally apply. Deviations and additions are explained below.

A gas phase remaining in the vinyl acetate separation 60, which is also designated here by 22, is first subjected to further compression 45a, and then fed to a carbon dioxide removal or corresponding process steps or system components 30. The carbon dioxide removal comprises, as before, an absorptive scrubbing. As described above, the carbon dioxide stream 33 obtained in the regeneration of the loaded absorbent (not explicitly shown) can be supplied, if desired, partially to the vinyl acetate synthesis and/or the process step(s) or system component(s) 20 in the form of a material stream 34 as a diluent in a quantity that is appropriate and can be adjusted easily at all times. Depending on the requirements, carbon dioxide removal can also comprise an optional alkali scrubbing (not explicitly shown) which serves to further reduce the carbon dioxide content in the gas stream with a carbon dioxide content already reduced in the absorptive scrubbing. After drying 80, this stream is conveyed to further distillative process steps and a further separation. These distillative process steps include in particular a C2 splitter 90 and an optional demethanizer 85, as already shown in FIG. 1.

In contrast to the embodiment shown in FIG. 1, the first product mixture 11 or a corresponding subsequent mixture 22 is not combined here with the second product mixture 21 or the corresponding subsequent mixture 22 before both are supplied to the process steps or system components 30—i.e., the carbon dioxide removal. Instead, a subsequent mixture 12a is fed in this case, after a compression 45, into a trace component removal 210, which in particular can comprise an acetylene hydrogenation, a carbon monoxide elimination and/or an oxygen removal, and into which a material stream 211, for example comprising additional water and/or oxygen, can be introduced. A subsequent mixture formed here, denoted by 31, is supplied to the process steps or system components 20, i.e., the vinyl acetate synthesis, and a product mixture formed there is treated as explained.

In FIG. 3, a process or a further system is shown in the form of a schematic flowchart, and is designated as a whole by 300. In principle, the explanations relating to FIGS. 1 and 2 and the process or system 100 or 200 apply here as well. Deviations and additions are explained in each case below.

The further compression 45a from FIG. 2 is omitted here, and—comparably to FIG. 2—the first and second subsequent mixture 12, 22 are combined before or in the compression 45. The formation of a third subsequent mixture 31 here additionally includes the removal of trace components as already explained in FIG. 2. This can be carried out as a crude gas treatment, in particular as shown schematically in FIG. 3 and as explained above. In this way, the carbon monoxide and/or acetylene contents can be reduced to a level that is not critical for the vinyl acetate synthesis in the process step(s) or system components 20. A partial stream 31a shown in dashed lines can be diverted upstream of the process step(s) or system components 30 and conducted directly to the vinyl acetate synthesis, and the fractions of ethane and carbon dioxide serve again in this case as the desired diluent which is necessary in practice. The further fractionation substantially corresponds to the process control according to FIG. 2. However, due to the positioning of a trace gas removal 210, a clear reduction of the oxygen fraction in the fourth subsequent mixture 32 can now be achieved. In particular, an excessive enrichment of oxygen in the top stream of the optional demethanizer 85 and the possible formation of a dangerous explosive atmosphere in this region can thereby be avoided.

In FIG. 4, a process or a further system is shown in the form of a schematic flowchart and is designated as a whole by 400. In principle, the explanations regarding FIGS. 1 to 3 and the process or the system 100 to 300 apply here as well. Deviations and additions are explained in each case below.

The crude gas removal 210 from FIG. 3 is omitted. Accordingly, the guidance of a partial stream 31a upstream of the carbon dioxide removal or the process steps or system components 30 directly to the vinyl acetate synthesis or the process steps or system components 20 is not technically expedient at this point, due to the expected contents of carbon monoxide and acetylene. The removal of carbon monoxide and oxygen traces then takes place by means of the demethanizer 85, which is thus necessarily to be provided in this embodiment of the invention. Due to the already mentioned possible formation of a dangerous, explosive atmosphere in the area of the top stream of the demethanizer 85, special additional aspects must be taken into account in the technical design in this area, in accordance with the relevant regulations and procedures known to those skilled in the art, as part of technical explosion protection.

In this embodiment of the invention, the feed stream of the C2 splitter 90 is subjected to a tail-end hydrogenation 410, which, with the addition of required hydrogen acetylene, selectively hydrogenates to form ethylene. Both the demethanizer 85 and the tail-end hydrogenation 410 can in each case be arranged independently of one another as shown in FIG. 4 upstream of the 90 or downstream of the C2 splitter 90 in the ethylene-enriched fraction C₂H₄. However, in any case, the demethanizer 85 must be arranged upstream of the tail-end hydrogenation 410, since carbon monoxide and oxygen in the tail-end hydrogenation 410 each have a negative influence.

1. A method for producing vinyl acetate, comprising:
in one or more first process steps:
subjecting ethane and oxygen to an oxidative catalytic dehydrogenation to obtain a first product mixture containing ethane, ethylene, water, acetic acid and carbon dioxide; and
in which, in one or more second process steps:
subjecting ethylene and acetic acid to a catalytic vinyl acetate synthesis to obtain a gaseous second product mixture containing ethylene, vinyl acetate, and carbon dioxide;
wherein at least a portion of the ethylene and at least a portion of the acetic acid which are subjected to the catalytic reaction in the one or more second process steps, are formed by at least a portion of the ethylene and at least a portion of the acetic acid from the first product mixture;
forming a first subsequent mixture containing ethane, ethylene and carbon dioxide using at least a portion of the first product mixture, wherein the formation of the first subsequent mixture comprises an at least partial removal of oxygen, carbon monoxide and/or acetylene, and further comprises subjecting at least a portion of the first product mixture and of the first subsequent mixture to a condensing deposition of water and acetic acid, to obtain a condensate fraction containing water and acetic acid, and wherein at least a portion of the acetic acid which is subjected to the catalytic vinyl acetate synthesis in the one or more second process steps is formed by at least a portion of the acetic acid from the condensate fraction;
forming a second subsequent mixture containing ethylene and carbon dioxide is formed using at least a portion of the second product mixture, wherein the formation of the second subsequent mixture comprises subjecting at least a portion of the second product mixture to a vinyl acetate separation to obtain a precursor fraction containing vinyl acetate and the second subsequent mixture;
combining at least most of the first subsequent mixture and the second subsequent mixture to form a third subsequent mixture containing ethane, ethylene and carbon dioxide;
subjecting at least a portion of the third subsequent mixture is subjected to one or more third process steps, wherein the one or more third process steps comprise a carbon dioxide removal, wherein a fourth subsequent mixture containing ethane and ethylene, and a carbon dioxide fraction, are formed in the one or more third process steps, and wherein at least a portion of the fourth subsequent mixture is subjected to a separation in which an ethane fraction and an ethylene fraction are formed; and
subjecting at least a part of the fourth subsequent fraction and/or at least a part of the ethylene fraction to a demethanization;
wherein:
at least a portion of the ethylene which is subjected to the catalytic vinyl acetate synthesis in the one or more second process steps is formed by at least a portion of the ethylene from the ethylene fraction, and/or at least a portion of the ethane which is subjected to the oxidative catalytic dehydrogenation in the one or more first process steps is formed by at least a portion of the ethane from the ethane fraction.

2. The method according to claim 1, wherein the carbon dioxide removal comprises a regenerative adsorptive carbon dioxide removal in which a scrubbing liquid is used which is loaded with carbon dioxide in the regenerative adsorptive carbon dioxide removal, and from which the carbon dioxide is expelled to obtain the carbon dioxide fraction.

3. The method according to claim 2, in which at least a portion of the carbon dioxide fraction is passed through the one or more second process steps as a diluent.

4. The method according to claim 3, in which the formation of the third subsequent mixture comprises subjecting at least a portion of the first subsequent mixture and at least a portion of the second subsequent mixture to a common compression.

5. The method according to claim 4, in which a portion of the acetic acid from the condensate fraction is used as an absorption medium in the vinyl acetate separation.

6. The method according to claim 5, in which at least a portion of the precursor fraction is subjected to a vinyl acetate purification in which a vinyl acetate fraction, a by-product fraction, and an acetic acid fraction are formed.

7. The method according to claim 6, in which the ethylene fraction is formed as an ethylene fraction containing up to 20 mol % ethane, and/or the ethane fraction is formed as an ethane fraction containing up to 20 mol % ethylene.

8. A system for the production of vinyl acetate, having:
one or more first system units which is or are designed to subject ethane and oxygen to an oxidative catalytic reaction to obtain a first product mixture containing ethane, ethylene, water, acetic acid and carbon dioxide;
one or more second system units which is or are configured to subject ethylene and acetic acid to a catalytic vinyl acetate synthesis to obtain a gaseous second product mixture containing ethylene, vinyl acetate, and carbon dioxide;
one or more third system units which is or are configured to subject at least a portion of a third subsequent mixture to a carbon dioxide removal, and which is or are configured to form a fourth subsequent mixture containing ethane and ethylene, and a carbon dioxide fraction, wherein at least a portion of the fourth subsequent mixture is subjected to a separation in which an ethane fraction and an ethylene fraction are formed;
wherein the system is configured to:
form at least a portion of the ethylene and at least a portion of the acetic acid which are subjected to the catalytic reaction in the one or more system units using at least a portion of the ethylene and at least a portion of the acetic acid from the first product mixture;
form a first subsequent mixture containing ethane, ethylene and carbon dioxide using at least a portion of the first product mixture, wherein the formation of the first subsequent mixture comprises an at least partial removal of oxygen, carbon monoxide and/or acetylene, and further comprises subjecting at least a portion of the first product mixture and of the first subsequent mixture to a condensing deposition of water and acetic acid, to obtain a condensate fraction containing water and acetic acid, and wherein at least a portion of the acetic acid which is subjected to the catalytic vinyl acetate synthesis in the one or more second process steps is formed by at least a portion of the acetic acid from the condensate fraction;
form a second subsequent mixture containing ethylene and carbon dioxide using at least a portion of the second product mixture, wherein the formation of the second subsequent mixture comprises subjecting at least a portion of the second product mixture to a vinyl acetate separation to obtain a precursor fraction containing vinyl acetate and the second subsequent mixture;
by combining at least most of the first subsequent mixture and the second subsequent mixture, to form a third subsequent mixture containing ethane, ethylene and carbon dioxide;
form at least a portion of the ethylene which is subjected to the catalytic vinyl acetate synthesis in the one or more second process steps by at least a portion of the ethylene from the ethylene fraction, and/or to form at least a portion of the ethane which is subjected to the oxidative catalytic dehydrogenation in the one or more first process steps by at least a portion of the ethane from the ethane fraction; and
subject at least a part of the fourth subsequent mixture and/or to subject at least a part of the ethylene fraction to a demethanization.

9. The system according to claim 8, wherein the carbon dioxide removal comprises a regenerative adsorptive carbon dioxide removal in which a scrubbing liquid is used which is loaded with carbon dioxide in the regenerative adsorptive carbon dioxide removal, and from which the carbon dioxide is expelled to obtain the carbon dioxide fraction.

10. The system according to claim 8, in which at least a portion of the carbon dioxide fraction is passed through the one or more second process steps as a diluent.

11. The system according to claim 8, in which the formation of the third subsequent mixture comprises subjecting at least a portion of the first subsequent mixture and at least a portion of the second subsequent mixture to a common compression.

12. The system according to claim 8, in which a portion of the acetic acid from the condensate fraction is used as an absorption medium in the vinyl acetate separation.

13. The system according to claim 8, in which at least a portion of the precursor fraction is subjected to a vinyl acetate purification in which a vinyl acetate fraction, a by-product fraction, and an acetic acid fraction are formed.

14. The system according to claim 8, in which the ethylene fraction is formed as an ethylene fraction containing up to 20 mol % ethane, and/or the ethane fraction is formed as an ethane fraction containing up to 20 mol % ethylene.

15. The method according to claim 1, in which at least a portion of the carbon dioxide fraction is passed through the one or more second process steps as a diluent.

16. The method according to claim 1, in which the formation of the third subsequent mixture comprises subjecting at least a portion of the first subsequent mixture and at least a portion of the second subsequent mixture to a common compression.

17. The method according to claim 1, in which a portion of the acetic acid from the condensate fraction is used as an absorption medium in the vinyl acetate separation.

18. The method according to claim 1, in which at least a portion of the precursor fraction is subjected to a vinyl acetate purification in which a vinyl acetate fraction, a by-product fraction, and an acetic acid fraction are formed.

19. The method according to claim 1, in which the ethylene fraction is formed as an ethylene fraction containing up to 20 mol % ethane, and/or the ethane fraction is formed as an ethane fraction containing up to 20 mol % ethylene.
